(19) United States
(12) Patent Application Publication
(10) Pub. No.: US 2015/0141518 A1
(43) Pub. Date: May 21, 2015

(54) WATER-MISCIBLE STABLE SOLUTION COMPOSITION FOR PHARMACEUTICALLY ACTIVE INGREDIENTS THAT ARE POORLY SOLUBLE IN WATER AND SUSCEPTIBLE TO CHEMICAL DEGRADATION

(71) Applicant: Emphascience, Inc., Pittsford, NY (US)

(72) Inventors: Rebanta Bandyopadhyay, Webster, NY (US); Susen Werle, Webster, NY (US)

(21) Appl. No.: 14/550,041

(22) Filed: Nov. 21, 2014

Related U.S. Application Data
(60) Provisional application No. 61/906,964, filed on Nov. 21, 2013.

(51) Int. Cl. A61K 47/10; A61K 9/08; A61K 31/167

(52) U.S. Cl. CPC A61K 47/10; A61K 31/167; A61K 9/08
USPC 514/629

(57) ABSTRACT

A method to improve the stability of unstable drug substances in solution is described. Such solution compositions are water -miscible, can be refrigerated, sterilized and even co-administered with other pharmaceutical products without causing precipitation of the active ingredient. A particular example of such a stable, water-miscible solution composition for paracetamol is disclosed.

### CROSS-REFERENCE TO RELATED APPLICATIONS

This application claims priority to and the benefit of the filing date of U.S. Provisional Patent Application Ser. No. 61/906,964 filed Nov. 21, 2013, the specification of which is hereby incorporated by reference in its entirety.

### BACKGROUND OF THE INVENTION

This invention relates to water-miscible stable solution compositions for an active pharmaceutical ingredient (API) that is susceptible to chemical degradation. Many APIs are preferably administered in the solution phase for quicker availability at the site of action and at adequate concentration. Accordingly, solution phases are injected when swift action is necessary or the active substance cannot be adequately absorbed by other routes where solid phases are administered, such as, by oral ingestion. Even for oral administration, solution compositions are sometimes preferred and can enhance the absorption of the active ingredient into the systemic circulation, when dissolution of the solid phase in the stomach and the intestines is slow and unpredictable. Compared to solid crystalline phases, challenges in the design of solution dosage forms include lack of sufficient solubility of the active in pharmaceutically acceptable solvent media and increased chemical instability.

The rate of any chemical reaction is increased when there is greater mobility of the reacting species, and hence solution phases can be more unstable and pose a challenge to formulate APIs that are susceptible to chemical degradation. The rate of degradation by oxidation, for example, can be further increased due to dissolved oxygen in the solution, presence of free radicals and metal ions that often catalyze such reactions. Hence, some common strategies to design stable injection formulations include the addition of antioxidants, free-radical scavengers, metal-chelating agents, and the reduction of dissolved oxygen in the solution phase and any gaseous environment directly in contact with it by replacing the oxygen with an inert gas, such as, nitrogen and argon.

For active medicinal substances that are particularly unstable, a more costly alternative is to freeze-dry the solution into a solid phase, which is reconstituted again into a solution prior to injection. However, reconstitution of a freeze-dried product and prompt administration can only be carried out by trained personnel to avoid compromising the safety and quality of the product, which adds to the cost and time in an urgent-care setting.

### SUMMARY OF THE INVENTION

One particular example is an aqueous injection drug product Ofirmev® containing paracetamol, which is indicated for the management of mild to moderate pain, for the management of moderate to severe pain with adjunctive opioid analgesics and for the reduction of fever. Although the solid phase of paracetamol is very stable at room temperature and under dry conditions, it degrades more easily in the presence of moisture and rapidly in solution to p-aminophenol, which subsequently undergoes oxidative degradation.

In particular the invention relates to sterile compositions including in one embodiment a sterile composition consisting essentially of: 0.1 to 23 weight percent paracetamol; and 77 to 98 weight percent polyethylene glycol; Wherein the polyetheylene glycol has an average molecular weight between 200 and 600.

In a second embodiment a sterile composition consisting essentially of: 0.1 to 23 weight percent paracetamol; 70 to 95 weight percent polyethylene glycol; and 2 to 20 weight percent water. Also, in a third embodiment A sterile composition consisting essentially of: 0.1 to 23 weight percent paracetamol; 77 to 98 weight percent polyethylene glycol; and 0.01 to 10 precent of a second active pharmaceutical agent chosen from the group consisting of various other analgesics, muscle relaxants, including hydrocodone, oxycodone, butalbital, caffeine, codeine, propoxyphene, tramadol, chlorpheniramine, pseudoephedrine, orphenadrine, methocarbamol, and salts or complexes thereof.

The solution composition for an unstable and poorly soluble API comprises dissolving the API, or its salt or its complex, either alone or in combination with other inactive and active ingredients, in one or more suitable water-miscible solvent(s) and adjusting the physical and chemical characteristics of the solution phase such that the rate of degradation is adequately lowered. More particularly, the selection of the solvent medium is designed to improve the solubility and stability of the API, such that the solution phase can be stored at temperatures lower or higher than that of ambient conditions and also be suitably diluted or mixed with other active and inactive ingredients without causing precipitation and, yet, when diluted, the osmotic pressure of the composition would be suitable for direct administration to a patient by injection, when applicable.

In a particular embodiment of the invention, a water-miscible stable solution composition containing the active pharmaceutical ingredient, paracetamol, also known as acetaminophen, and salts or complexes thereof, either alone or in combination with other active and inactive pharmaceutical ingredients is described that is stable through the process of manufacturing, sterilization and storage, and can also be administered to humans or animals for therapeutic purposes following suitable dilution and dose adjustment, as necessary for the treatment of pain and fever.

The solution composition claimed in this patent does not require the reduction of dissolved oxygen in the solution or in its surrounding environment, does not require the addition of buffers or controlled pH, does not require free-radical scavengers and antagonists, does not contain antioxidants, metal chelators, such as ethylenediamine tetra acetic acid (EDTA) or salts thereof, other organic acids or mannitol and, yet, remains physically and chemically stable for extended periods of time. As a further advantage, the concentration of paracetamol in the current formulation can be significantly higher than the solubility of the API in water, thus making it possible to contain the maximum unit-dose in a volume that is significantly less than that of marketed products. Despite such higher concentration of paracetamol, the novel composition claimed in the patent is compatible with many other pharmaceutical products and APIs, can be sterilized, stored under refrigerated conditions, or diluted to isotonic concentrations, or mixed with formulations of other active ingredients without causing precipitation.

### BRIEF DESCRIPTION OF THE DRAWINGS

FIG. 1 demonstrates the chemical stability of the novel solution composition of paracetamol in comparison to a commercially available solution phase product under high temperature (80° C.) stressed-storage conditions. T

FIG. 2 illustrates various embodiments of the invention.

### DETAILED DESCRIPTION OF INVENTION

Degradation leading to chemical and physical changes can negatively impact the safety and efficacy of a pharmaceutical product. For example, changes to the chemical structure of an active molecule by a chemical reaction can result in loss of its pharmacological activity (loss of potency) and/or increase in toxicity due to undesirable side effects from substances with different chemical structures. Similarly, a physical change, such as, decrease in viscosity of a suspension product can cause quicker settling of the suspended phase leading to lack of dose uniformity. Unfortunately, a vast majority of active pharmaceutical ingredients are susceptible to physical and chemical changes during manufacture, storage or use under the influence of environmental factors, such as, temperature, light, moisture and atmospheric oxygen. The rate of change is usually accelerated when the product is a solution, and the impact of pH, concentration of the active, as well as inactive ingredients in the formulation, manufacturing and packaging components that come in direct contact with the product also become important.

The overall rate of chemical degradation of paracetamol in solution follows first order kinetics and is significantly accelerated in extremes of pH 9 or 5 2) and at higher temperatures, doubling with every 5° C. rise in temperature from 25° C. to being almost an order of magnitude higher at 45° C¹. Hence, paracetamol in Ofirmev®is stabilized by the addition of cysteine hydrochloride as an antioxidant, dibasic sodium phosphate as a buffer to adjust the pH to approximately 5.5 and by manufacturing and packaging the product under an inert gas atmosphere to reduce the dissolved oxygen content to less than 2 parts per million (ppm). To be administered directly as an infusion, Ofirmev® also contains a tonicity adjuster, mannitol, which can also act as a metal chelator under certain conditions. Aside from the risks of higher chemical degradation at elevated temperatures, the poor solubility of paracetamol in water² can cause its precipitation from Ofirmev® at temperatures below 15° C. Care needs to be taken to avoid exposing this solution product to colder temperatures, such as refrigeration, or of mixing and co-administering with other products to avoid precipitation of one or more of the poorly soluble actives, such as, diazepam and chlorpromazine hydrochloride. Hence, a solution composition for unstable drug substances, such as paracetamol, that is physically and chemically stable, is water-miscible and can be sterilized by heat sterilization for administration as an injection would be a definite advantage over the current products available for therapy. The solution composition for an unstable and poorly soluble API comprises dissolving the API, or its salt or its complex, either alone or in combination with other inactive and active ingredients, in one or more suitable water-miscible solvent(s) and adjusting the physical and chemical characteristics of the solution phase such that the rate of degradation is adequately lowered. More particularly, the selection of the solvent medium is designed to improve the solubility and stability of the API, such that the solution phase can be stored at temperatures lower or higher than that of ambient conditions and also be suitably diluted or mixed with other active and inactive ingredients without causing precipitation and, yet, when diluted, the osmotic pressure of the composition would be suitable for direct administration to a patient by injection, when applicable. In a particular embodiment of the invention, a water-miscible stable solution composition containing the active pharmaceutical ingredient, paracetamol, also known as acetaminophen, and salts or complexes thereof, either alone or in combination with other active and inactive pharmaceutical ingredients is described that is stable through the process of manufacturing, sterilization and storage, and can also be administered to humans or animals for therapeutic purposes following suitable dilution and dose adjustment, as necessary for the treatment of pain and fever. The solution composition claimed in this patent does not require the reduction of dissolved oxygen in the solution or in its surrounding environment, does not require the addition of buffers or controlled pH, does not require free-radical scavengers and antagonists, does not contain antioxidants, metal chelators, such as ethylenediamine tetra acetic acid (EDTA) or salts thereof, other organic acids or mannitol and, yet, remains physically and chemically stable for extended periods of time. As a further advantage, the concentration of paracetamol in the current formulation can be significantly higher than the solubility of the API in water, thus making it possible to contain the maximum unit-dose in a volume that is significantly less than that of marketed products. Despite such higher concentration of paracetamol, the novel composition claimed in the patent is compatible with many other pharmaceutical products and APIs, can be sterilized, stored under refrigerated conditions, or diluted to isotonic concentrations, or mixed with formulations of other active ingredients without causing precipitation.

A method to reduce the rate of change in solution is to select a solvent or a mixture of solvents that offer the least reactive environment for the active pharmaceutical ingredient, taking into account the elemental composition and specific structural features of the entities so that the interaction between reacting entities is minimized. Selection of an optimum solvent system can be supplemented by the addition of other inactive ingredients that competitively reduce the propensity for change. Physical characteristics of the solution, such as, viscosity may also be increased to decrease the molecular mobility and diffusion of reactive species through the medium, thus reducing the rate of reaction. However, to facilitate manufacturing and filtration for injections, and to counter the inconvenience to the care giver or the risk of injury to patients, the product has to be converted back to a low-viscosity, free-flowing liquid phase, when needed.

In one example involving a model drug, paracetamol—a centrally acting analgesic that is susceptible to rapid oxidation and hydrolysis in solution even under ambient storage conditions, the method involves dissolving the drug in a suitable water-miscible, but non-aqueous solvent at a high concentration so that the propensity of hydrolysis in the presence of water is reduced. Additionally, since auto-oxidation in the presence of atmospheric oxygen or that dissolved in the solution medium can be accelerated by the presence of free radicals and/or metal ions, the current method involves dissolving paracetamol in a non-aqueous medium that is miscible in water but is capable of restricting the access to atmospheric oxygen, moisture and other facilitators of hydrolysis and oxidation. Once diluted, the product attains acceptable drug concentration, osmotic pressure and physical properties, such as viscosity, for direct intravenous administration.

Paracetamol is poorly soluble in water, but has better solubility in short-chain alcohols and polyols, such as, methanol, ethanol, propanol, glycerol, various glycols, and is also soluble in many other moderately polar solvents, such as, tetrahydrofuran, acetone, diethylamine, dimethyl sulfoxide, and N,Ndimethylformamide. However, since many of the above solvents are not suitable for pharmaceutical use or cannot be injected at the required concentrations due to toxicity, the choice of solvent is restricted to those that can be safely administered following dilution.

The prior art has taught methods of creating stable pharmaceutical solution compositions for paracetamol in water and a mixture of, among others, alkyl polyhydroxylated compounds that can include polyethylene glycols of various molecular weights, but requires the addition of a buffer and at least one member of the group comprising a free radical scavenger and a radical antagonist.

In one particular example of the present invention, paracetamol is dissolved in a pharmaceutically acceptable polyol, such as, glycerol, propylene glycol (PG) or polyethylene glycol (PEG) or mixtures thereof at higher concentrations to form a stable, concentrated solution, which can then be diluted with water or other water-based, commonly used injectable diluents, such as, water for injection, isotonic 0.9% NaCl solution in water, or 5% dextrose solution, or Lactated Ringer's solution to reduce the concentration to 10 mg/mL of paracetamol, which is comparable to that in currently marketed products without causing precipitation or changes in chemical stability.

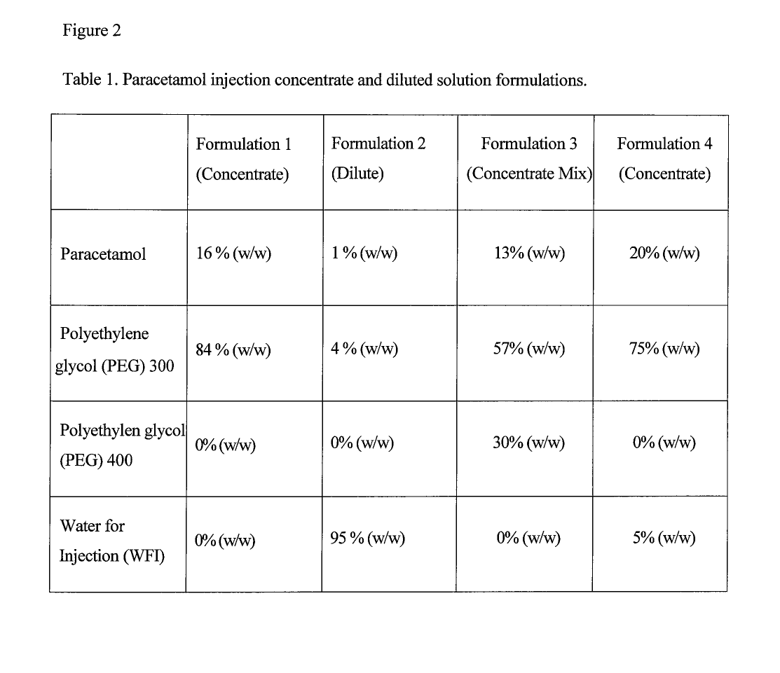

An even more specific application of this method involves dissolving the commonly recommended maximum unit dose for adults, i.e. 1000 mg of paracetamol, in approximately 5 mL of pure PEG 300. The concentrated solution can be diluted with Water-for-Injection (WFI) or other suitable diluents for administration by intravenous infusion. The concentration of paracetamol can range from 0.1% to 20% (w/w) and optionally, tonicity adjusters can be added to the formulation to allow direct intravenous injection without much discomfort to the patient. Two simple examples of stable concentrated and dilute formulations of paracetamol are shown in FIG. 2.

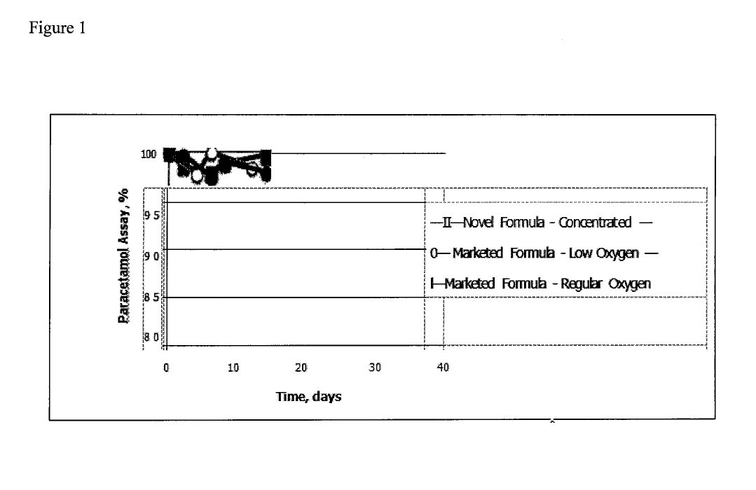

In the resulting concentrated solution, paracetamol degrades to less than 5% in over 30 days when stored at 80° C. and is expected to be stable for over 3 years at ambient storage conditions (room temperature) without the need to control/reduce the oxygen content of the environment or that in solution, without the addition of buffers or controlled pH, without any free-radical scavengers, antagonists, antioxidants, metal chelators such as ethylenediamine tetra acetic acid (EDTA) or salts thereof, other organic acids and mannitol (FIG. 1). Less than 10% of paracetamol is degraded over a month in the diluted formulation, even when the solution is stored at 80° C. The comparable marketed product for paracetamol injection, on the other hand, imparts acceptable stability to paracetamol only when the dissolved oxygen content in the solution is reduced and in the presence of buffers, free-radical scavenger and antioxidant.

To facilitate proper mixing and dissolution in a relatively viscous medium, such as polyethylene glycol, the manufacturing process may include heating the solvent up to 50° C. to reduce the viscosity to facilitate quicker dissolution of the active.

FIG. 1. The stability of solution phase paracetamol at high temperature (80° C.) stressed-storage conditions over a period of a month in the novel composition compared to that in the current injectable product in the market with and without reduced oxygen content.

In another more specific example of the application of this method, paracetamol can be dissolved in pure PEG or in various combinations of PEG and water, such that the end-product following dilution is suited for direct intravenous injection.

Although all molecular weights of polyethylene glycol (PEG) that are liquid at room temperature can be used, the solution of paracetamol in PEG 300, in particular, is preferable, since relatively high proportions of PEG 300 can be administered by intravenous injection, and it offers the optimum balance of viscosity, solubility, and manufacturability of the product without compromising its stability.

In the given example, the concentration of paracetamol is such that when diluted to 10 mg/mL concentration with water for injection, the diluted solution becomes isotonic and can be directly administered as an intravenous injection.

One additional advantage of this solution composition is that it can accommodate a high concentration of paracetamol of at least up to 225 mg/mL, which is significantly higher than the solubility of paracetamol in water (ca. 13 mg/mL) at room temperature and, yet, can be refrigerated and diluted to lower concentrations with pharmaceutically acceptable diluents for the purpose of storage, shipping, co-administering the product with various other therapeutic agents without causing precipitation or other undesirable physical and chemical changes to the API. These sterile composition consisting essentially of: 0.1 to 23 weight percent paracetamol; and 77 to 98 weight percent polyethylene glycol; wherein the polyetheylene glycol has an average molecular weight between 200 and 500. Another embodiment might be a composition consisting essentially of: 0.1 to 23 weight percent paracetamol; 70 to 95 weight percent polyethylene glycol; and 2 to 20 weight percent water; or alternately the composition might consist essentially of: 0.1 to 23 weight percent paracetamol; 77 to 98 weight percent polyethylene glycol; and 0.01 to 10 precent of a second active harmaceutical agent chosen from the group consisting of hydrocodone, oxycodone, butalbital, caffeine, codeine, propoxyphene, tramadol, chlorpheniramine, pseudoephedrine, orphenadrine, methocarbamol, and salts or complexes thereof.

The invention includes solution compositions consisting essentially of 0.1 to 23 weight percent paracetamol; and 77 to 99 weight percent polyethylene glycol where the polyethylene glycol is a liquid in the temperature range of 20-25° C. that can be sterilized by the application of heat or any other conventional sterilization method after the components are combined. The average molecular weight of said polyethylene glycol is between 200 and 600 or any combination of average molecular weights. In other embodiments the paracetamol comprises more than 15 weight percent of the composition.

The invention provides the benefit of being essentially free of any undesirable or toxic degradation products of paracetamol, including 4-aminophenol and hydroquinone even after storage at 40° C. for at least 3 months or more and the chemical stability of acetaminophen is not negatively impacted by the presence of dissolved oxygen as under normal ambient conditions and can be stored over a temperature range of 2-8° C. for at least 6 months without precipitation of paracetamol solid phase. These compositions can be diluted with a suitable injection medium without causing precipitation of the active, paracetamol. Thus they can be administered by bolus injection or as an infusion following dilution.

In other embodiments the compositions consisting essentially of 0.1 to 23 weight percent paracetamol; 65 to 95 weight percent polyethylene glycol; and up to 20 weight percent water. These embodiments can be sterilized by the application of heat or any other conventional sterilization method after the components are combined and provide high concentrations of paracetamol in excess of 15 weight percent of the composition.

What is claimed is:

1. A solution composition consisting essentially of:
0.1 to 23 weight percent paracetamol; and
77 to 99 weight percent polyethylene glycol;
Wherein the polyethylene glycol is a liquid in the temperature range of 20-25° C.

2. The composition of claim 1 wherein the composition can be sterilized by the application of heat or any other conventional sterilization method after the components are combined

3. The solution composition of claim 1 wherein the molecular weight of said polyethylene glycol is between 200 and 600 or any combination thereof.

4. The solution composition of claim 1 wherein paracetamol comprises more than 15 weight percent of the composition.

5. The solution composition of claim 1, which is essentially free of any undesirable or toxic degradation products of paracetamol, including 4-aminophenol and hydroquinone even after storage at 40° C. for at least 3 months or more

6. The solution composition of claim 1, in which the chemical stability of acetaminophen is not negatively impacted by the presence of dissolved oxygen as under normal ambient conditions

7. The solution composition of claim 1, which can be stored over a temperature range of 2-8° C. for at least 6 months without precipitation of paracetamol solid phase

8. The solution composition of claim 1, which can be diluted with a suitable injection medium without causing precipitation of the active, paracetamol

9. The solution composition of claim 1, which can be administered by bolus injection or as an infusion following dilution.

10. A solution composition consisting essentially of:
0.1 to 23 weight percent paracetamol;
65 to 95 weight percent polyethylene glycol; and
up to 20 weight percent water.

11. The composition of claim 1 wherein the composition can be sterilized by the application of heat or any other conventional sterilization method after the components are combined

12. The solution composition of claim 1 wherein the molecular weight of said polyethylene glycol is between 200 and 600 or any combination thereof.

13. The solution composition of claim 1 wherein paracetamol comprises more than 15 weight percent of the composition.

14. The solution compositions of claims 1, which do not cause precipitation of other solution drug products, such as, chlorpromazine hydrochloride and diazepam, when mixed together and co-administered.

15. The solution compositions of claims 10, which do not cause precipitation of other solution drug products, such as, chlorpromazine hydrochloride and diazepam, when mixed together and co-administered
